(19) United States
(12) Patent Application Publication
(10) Pub. No.: US 2010/0094249 A1
(43) Pub. Date: Apr. 15, 2010

(54) WOUND TREATMENT APPARATUS AND METHODS

(76) Inventor: Ty Caswell, Zimmerman, MN (US)

Correspondence Address: HOLLAND & HART LLP, 60 E. South Temple, Suite 2000, P.O. Box 11583, Salt Lake City, UT, 84110, US

(21) Appl. No.: 12/576,976

(22) Filed: Oct. 9, 2009

Related U.S. Application Data
(60) Provisional application No. 61/104,208, filed on Oct. 9, 2008.

(51) Int. Cl. A61M 31/00; A61M 1/00

(52) U.S. Cl. 604/500; 604/257; 604/246; 604/131; 604/151; 604/140

(57) ABSTRACT

An wound treatment device includes a housing and a treatment tip. The housing retains a volume of treatment fluid. The treatment tip includes a recess, a peripheral edge surrounding the recess, and a fluid channel in view of communication with the recess and the volume of treatment fluid. The recess may be sized to enclose at least a portion of a wound such as an acne member on a person's skin. The recess is sized to retain a volume of the wound treatment fluid when the peripheral edge is sealingly engaged with a skin surface surrounding at least a portion of the wound.

### CROSS-REFERENCE TO RELATED APPLICATION

This application claims the benefit of the filing date of U.S. Provisional Application No. 61/104,208, filed Oct. 9, 2008, and entitled ACNE TREATMENT APPARATUS AND METHODS, the disclosure of which is incorporated, in its entirety, by reference.

### TECHNICAL FIELD

The present disclosure generally relates to the treatment of wounds and related devices for such treatment.

### BACKGROUND

Many types of wounds can benefit from application of medications. The shape, size and location of a wound as well as the type of wound and any associated diseases or bacteria that are present can all effect how a wound is most effectively treated. Wounds can generally be categorized into one of the following types:
Abrasions. Also called scrapes, they occur when the skin is rubbed away by friction against another rough surface (e.g., rope bums and skinned knees).
Avulsions. Occur when an entire structure or part of it is forcibly pulled away, such as the loss of a permanent tooth or an ear lobe. Explosions, gunshots, and animal bites may cause avulsions.
Contusions. Also called bruises, these are the result of a forceful trauma that injures an internal structure without breaking the skin. Blows to the chest, abdomen, or head with a blunt instrument (e.g. a football or a fist) can cause contusions.
Crush wounds. Occur when a heavy object falls onto a person, splitting the skin and shattering or tearing underlying structures.
Cuts. Slicing wounds made with a sharp instrument, leaving even edges. They may be as minimal as a paper cut or as significant as a surgical incision.
Lacerations. Also called tears, these are separating wounds that produce ragged edges. They are produced by a tremendous force against the body, either from an internal source as in childbirth, or from an external source like a punch.
Missile wounds. Also called velocity wounds, they are caused by an object entering the body at a high speed, typically a bullet.
Punctures. Deep, narrow wounds produced by sharp objects such as nails, knives, and broken glass

Acne is a common disease characterized by various types of wounds. The areas affected typically are areas of the skin were sebaceous glands are largest, most numerous, and most active. Acne typically occurs during the teenage years due to hormonal changes, but occasionally develops as early as the age of nine years and sometimes extends well into adult ages.

In its mildest from, acne is a more or less superficial disorder characterized by slight, spotty skin irritations. In such cases, ordinary skin hygiene is typically a satisfactory treatment. In the more inflammatory types of acne, however, pustules, infected cysts, and in extreme cases, canalizing, inflamed and infected sacs and other wounds appear. Without effective treatment, these lesions may become extensive and leave permanent, disfiguring scars.

A need exists for improved wound treatment devices and methods, in particular related to wounds associated with acne.

### SUMMARY

The present disclosure relates to wound treatment devices and methods generally. One aspect of the present disclosure is directed to a method of treating a wound with a treatment fluid. At least a portion of the wound may be positioned below an outer skin surface. The method includes creating a seal on the outer skin surface, delivering the treatment fluid into the wound, and maintaining the seal to retain the treatment fluid within the wound.

A treatment device may be used to perform at least some of the method steps. An example treatment device includes a treatment tip configured to provide the seal. Delivering the treatment fluid to the wound may include delivering the treatment fluid through the treatment tip.

Another example treatment device may include a container. The container retains a volume of the treatment fluid. Delivering the treatment fluid may include compressing a portion of the container to increase a pressure condition of the treatment fluid.

A further example treatment device includes a fluid conduit and a treatment tip. Creating the seal may include contacting the treatment tip against the outer skin surface. Delivering the treatment fluid into the wound may include delivering the treatment fluid through the fluid conduit to the treatment tip.

The method may also include mixing the treatment fluid with a propellant prior to delivering the treatment fluid to the wound. Delivering the treatment fluid may include controlling flow of treatment fluid with a one-way valve member. The method may be related specifically to treating a wound that is an acne member, and the treatment fluid may be a solution comprising an antibiotic.

Another aspect of the present disclosure relates to an wound treatment device. The wound treatment device may include a housing and a treatment tip. The housing is configured to retain wound treatment fluid. The treatment tip includes a recess, a peripheral edge surrounding the recess, and a fluid channel in fluid communication with the recess and the volume of wound treatment fluid. The recess is sized to retain a volume of the wound treatment fluid when the peripheral edge is sealingly engaged with a skin surface surrounding at least a portion of a wound.

A further aspect of the present disclosure relates to a method of treating a skin acne member. The method includes providing a source of acne treatment fluid and a dispensing device having a treatment tip, sealingly engaging the treatment tip against a user's skin surface surrounding at least a portion of the acne member, thereafter delivering the acne treatment fluid to the treatment tip and into contact with the acne member, and penetrating the acne member with the acne treatment fluid.

The above summary of the present invention is not intended to describe each embodiment or every implementation of the present invention. The attached FIGS. and the detailed description that follow more particularly exemplify examples embodiments including inventive aspects of the present disclosure.

### BRIEF DESCRIPTION OF THE DRAWINGS

FIG. 1 is a schematic cross-sectional side view of an example wound treatment system in an upright orientation according to the present disclosure.

FIG. 2 is a schematic cross-sectional side view of the wound treatment system shown in FIG. 1 facing a wound member on a skin surface.

FIG. 3 is a schematic cross-sectional side view of the wound treatment system shown in FIG. 2 with a treatment tip of the system in engagement with the skin surface and enclosing the wound member.

FIG. 4 is a schematic cross-sectional side view of another example wound treatment system having a metering device associated therewith in accordance with the present disclosure.

FIG. 5 is a schematic side view of another example wound treatment system in accordance with the present disclosure.

FIGS. 6A-C are schematic front views of several example treatment tip configurations in accordance with the present disclosure.

FIG. 7 is a flow diagram illustrating steps of an example treatment method in accordance with the present disclosure.

FIG. 8 is a perspective view of another example wound treatment system in accordance with the present disclosure.

FIG. 9 is a real perspective view of wound treatment system shown in FIG. 9.

FIG. 10 is a top view of the wound treatment system shown in FIG. 8.

FIG. 11 is a cross-sectional view of the wound treatment system shown in FIG. 10.

FIG. 12 is a perspective view of the wound treatment system shown in FIG. 1 with a removal cap of the system removed.

FIG. 13 is a perspective view of another example wound treatment system in accordance with the present disclosure.

FIG. 14 is a side view of a wound treatment system shown in FIG. 13.

FIG. 15 is a cross-sectional view of the wound treatment system shown in FIG. 14.

FIG. 16 is an exploded perspective view of the wound treatment system shown in FIG. 13.

FIG. 17 is a perspective view of another example of wound treatment system in accordance with the present disclosure.

FIG. 18 is an exploded perspective view of the wound treatment system shown in FIG. 17.

FIG. 19 is a cross-sectional view of the wound treatment system shown in FIG. 17.

FIG. 20 is a perspective view of another example wound treatment system in accordance with the present disclosure.

FIG. 21 is a side view of a wound treatment system shown in FIG. 20.

FIG. 22 is a cross-sectional view of the wound treatment system shown in FIG. 21.

FIG. 23 is an exploded perspective view of the wound treatment system shown in FIG. 20.

FIG. 24 is a perspective view of another example wound treatment system in accordance with the present disclosure.

FIG. 25 is a side view of a wound treatment system shown in FIG. 24.

FIG. 26 is a cross-sectional view of the wound treatment system shown in FIG. 25.

FIG. 27 is an exploded perspective view of the wound treatment system shown in FIG. 24.

FIG. 28 is a perspective view of another example wound treatment system in accordance with the present disclosure.

FIG. 29 is a side view of a wound treatment system shown in FIG. 28.

FIG. 30 is a cross-sectional view of the wound treatment system shown in FIG. 29.

FIG. 31 is an exploded perspective view of the wound treatment system shown in FIG. 28.

### DETAILED DESCRIPTION

The present disclosure is directed to improved wound treatment devices and related systems and methods of treating wounds, such as skin acne. The examples disclosed herein are primarily directed to devices and methods for applying a wound treatment fluid (e.g., liquid, aerosol, gel, semi-gel, or foam). The wound treatment fluid can be applied topically. In some arrangements, the wound treatment fluid can be applied so as to penetrate or partially penetrate the skin or a wound member. While examples given herein may be directed to specific types of wounds and particular devices and methods for treating a specific wound, the general principles are applicable to many wounds and many treatment devices and methods.

The example wound treatment devices disclosed herein may be used for treating any number of different types of wounds. The wounds may be exposed on an outer surface of a body or may be located within the body beneath the outer surface. For example, the wound treatment device could be used during surgery to apply a treatment fluid to a wound, organ, or tissue positioned inside the body that is exposed during the surgical procedure.

In addition to traditional in-home, personal use of the wound treatment device (e.g., for treatment of acne members as described in more detail below), the examples disclosed herein may be adapted for use in or included in first aid kits, emergency medical response applications such as ambulances and in-the-field military situations, medical triage centers, medical clinics, emergency rooms, and hospitals generally. Furthermore, some embodiments may be configured for veterinary uses in treating animals (e.g., mammals, birds, reptiles, etc.) rather than humans.

One aspect of the present disclosure relates to the treatment of wounds in the form of skin acne. Skin acne is typically treated with medications that are either applied topically to the acne member in the form of a paste or like solution, or medications taken orally. Some types of medications that must be applied topically to the acne member have a consistency that makes it difficult to retain the solution on the acne member without the solution immediately “running off” the skin surface or otherwise maintaining contact with the acne member for a period of time necessary for the solution to penetrate into the acne member. The devices, systems and methods described herein facilitate engagement of a topically applied acne treatment fluid with an acne (i.e., an wound) for an extended time period that permits penetration of the fluid into the acne member. In some cases, the acne treatment fluid is applied in a pressurized state that can both increases penetration of the fluid into the acne member and reduce the overall time required for penetration of the fluid into the acne member.

The term “pressurized” as used herein may be defined in several ways. According to one definition, “pressurized” is a pressure level, state or condition for the treatment fluid that is higher than a pressure level, state, or condition within the wound (e.g., an acne member) being treated. The term “pressurized” may be defined as a pressure level, state or condition that is higher than at a rest pressure level, state or condition. For example, a container holding a volume of fluid may maintain a rest pressure state or condition when no external forces are acting upon the container, and the volume of fluid may be changed to a pressurized state or higher pressure level upon squeezing the container. A “pressurized” level, state or condition may be defined relative to atmospheric pressure, such as being higher than atmospheric pressure. According to another definition, “pressurized” is merely the pressure level, state or condition needed to create fluid flow, such as flow through a conduit, out of a nozzle or other structure having an opening, or through a permeable or semi-permeable structure (i.e., a skin surface).

Another aspect of the present disclosure relates to a method of treatment in which the treatment device is brought into engagement with the skin surface and enclosing an acne member. A volume of acne treatment fluid is brought into contact with the acne member and maintained in contact for a minimum amount of time such as, for example, at least about 2 to about 10 seconds. The acne treatment fluid may be delivered to the skin surface in a pressurized state. Thereafter, the treatment device is removed from engagement with the skin surface and any excess amounts of the acne treatment fluid that has not penetrated into the acne member can be wiped clean from the skin surface. The method can be repeated as needed to ensure that a portion of the acne treatment fluid has penetrated into the acne member.

The amount of time needed to provide delivery of a treatment fluid into or onto a wound may depend on a number of variables. For example, when a wound is open or includes an opening into the wound, the treatment fluid may more easily flow into the wound, thus involving less time for delivery. In other arrangements wherein the wound is closed or substantially closed, more time may be needed to deliver the treatment fluid. A “predetermined” amount of time to deliver the treatment fluid may be recommended for a given type of wound.

The predetermined amount of time may be the amount of time needed for optimum performance (i.e., best chance of delivery occurring on a first attempt) based on experimentation and testing.

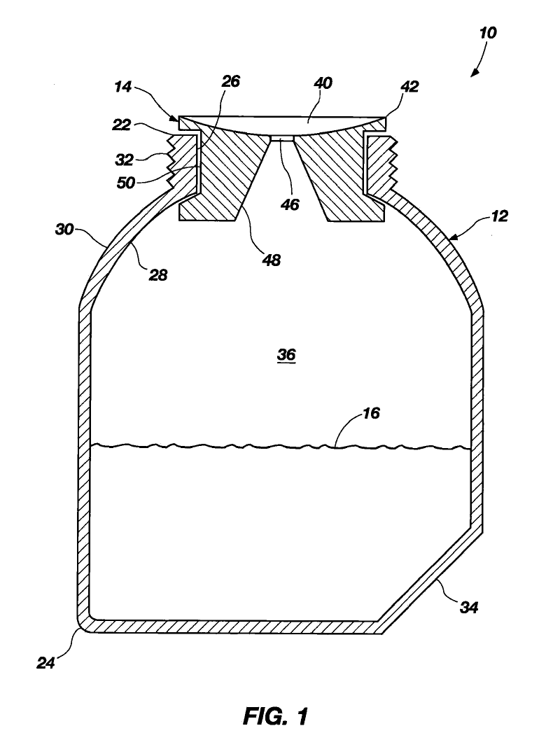

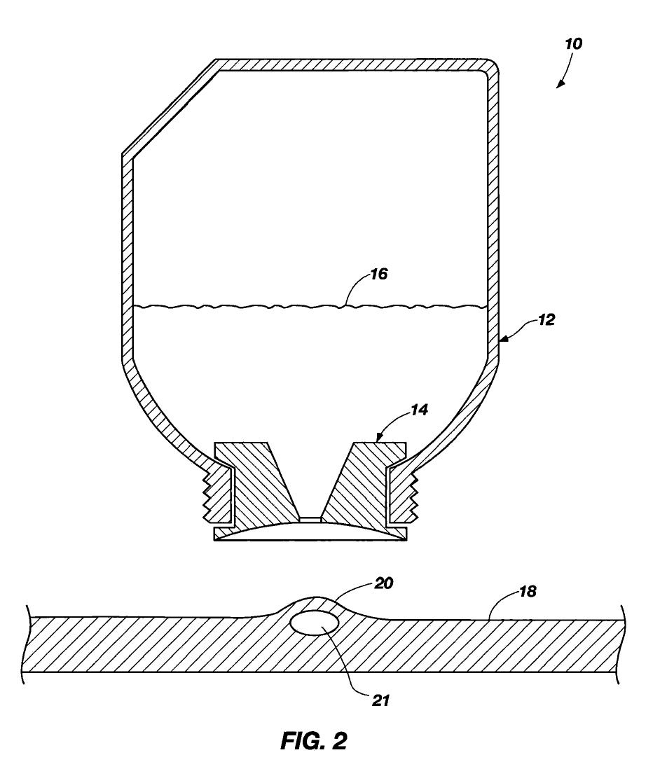

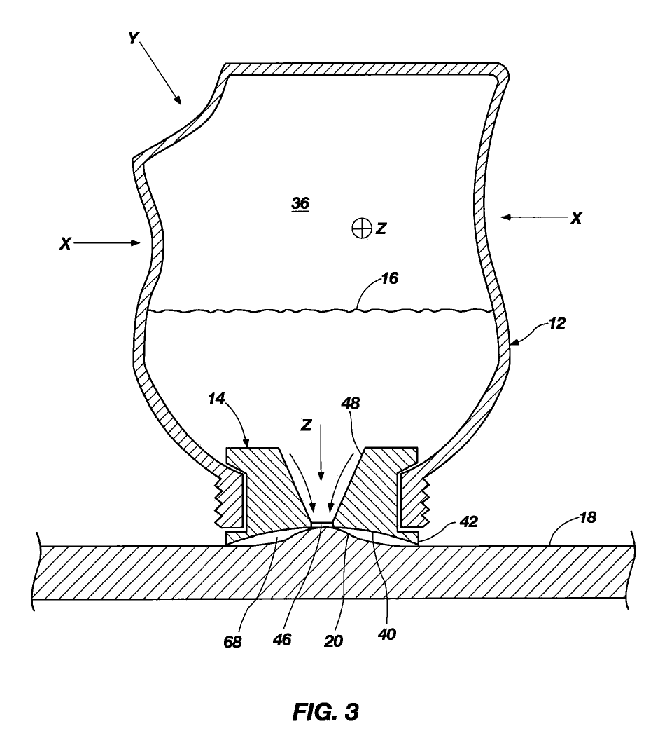

An example treatment device 10 and related methods of treating an acne member are now described as referenced FIGS. 1-3. The treatment device 10 includes a container 12 and a treatment tip 14. Container 12 is sized to retain a volume of a treatment fluid 16 for treatment of an acne member 20 that is located on or under a the skin surface 18 (see FIGS. 2 and 3). The acne member may include a liquid or infected portion 21 that is positioned beneath the skin surface 18. This portion 21 may be accessible for treatment by penetrating the treatment fluid through the skin surface 18 into the portion 21. Alternatively, the acne member 20 may be burst to create a fluid flow path through the skin surface 18 into the portion 21.

The container 12 includes a top end 22, a bottom end 24, an opening 26 defined at the top end 22, an inner surface 28, and an outer surface 30. Threads or other connecting features may be positioned on the outer surface 30 at the top end 22 (e.g., adjacent to the opening 26). The threads 32 can be used to mount a cap to the container 12 that encloses the treatment tip 14.

The container 12 defines an internal volume 36 that retains the treatment fluid 16. The internal volume 36 can have a size in the range of, for example, about 1 mL to about 100 mL, and preferably in the range of about 1 mL to about 20 mL. In some examples, the internal volume 36 can be sized smaller, such as, for example, in the range of about 0.1 to about 5 mL. Such smaller sized containers can be used as, for example, single use or limited use devices as compared to larger containers that retain a more significant volume of treatment fluid for multiple treatments. Any container size can be used depending on the intended use of the device 10.

The container 12 can comprise a compliant or semi-compliant material that facilitates compression or otherwise inward flexing of portions of the container. The container 12 can further include a cutaway or otherwise angled portion 34 that helps in grasping the container 12 by a user applying pressure to the device against the user skin surface, or applying the compression force that flexes the container sidewalls inward. Compressing portions of the container 12 can pressurize the treatment fluid 16 retained therein to force a portion of the treatment fluid out of the treatment tip 14 in a pressurized state. Supplying the treatment fluid 16 out of the treatment tip 14 in a pressurized state is one aspect of the treatment methods as will be described in further detail below.

Supplying the treatment fluid 16 in a pressurized state out of the treatment tip 14 can be accomplished by alternative methods in addition to merely applying a compression force to portions of the container 12. For example, the treatment device 10 can include a source of pressurized propellant (e.g., a bag-on-valve (BOV) pressure system) or can include features that generate a pressurized state within the container 12 upon activation by the user (e.g., activation of a propellant compressor or activation of a chemical reaction that generates a pressurized propellant). An example BOV system is disclosed in U.S. Pat. No. 6,874,659, which is incorporated herein by reference.

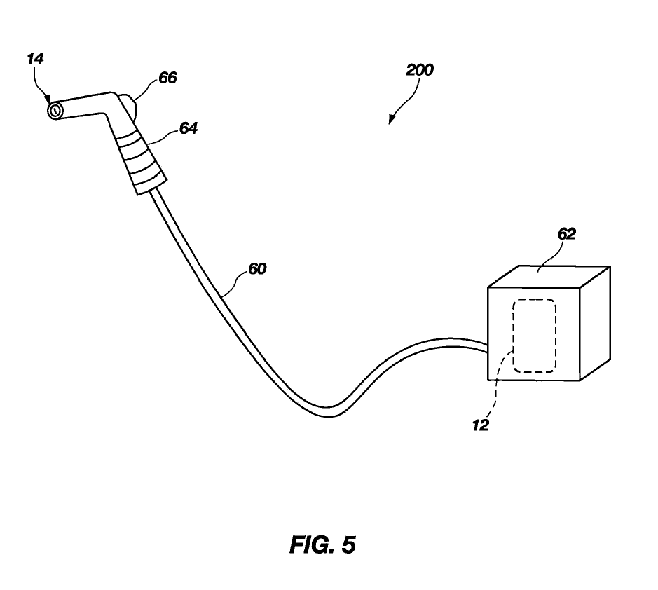

Alternatively, the treatment device 10 can be coupled to a remote source of pressurized propellant such as via a pressurize line mounted to a portable pressure generating device. The acne treatment system 200 shown and described below with reference to FIG. 5 is one example of such an alternative system or device with a remote pressure source. Systems that provide the acne treatment fluid in a pre-pressurized state can in at least some instances be easier to operate by the user and can reduce the time necessary to treat the acne member.

The treatment tip 14 includes a contact surface 40 having a peripheral edge 42. The contact surface 40 may be recessed relative to the peripheral edge 42 and may be referred to as a recessed surface 40. The contact surface 40 may be configured as a concave structure. A concave shaped contact surface can be particularly easy to clean out prior to or after use of the device 10 using, for example, a finger tip or a wipe (e.g., a towel or tissue) to remove contaminates and debris. In some arrangements, the contact surface 40 is substantially planar or includes at least one planar surface. In still further arrangements, at least a portion of the contact surface 40 includes a protrusion such as a convex shaped portion.

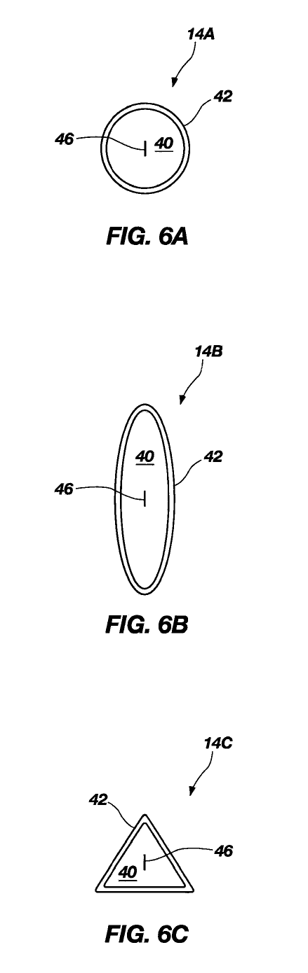

The contact surface 40 and peripheral edge 42 can have many different shapes and sizes. FIGS. 6A-C are front views of treatment tips 14A-C illustrating circular, oval, and triangular shaped treatment tip contact surfaces 40, respectively.

A valve opening 46 can be positioned on the contact surface 40 to provide fluid communication between the contact surface 40 and the internal volume 36 of the container 12. A fluid channel 48 can be defined in the treatment tip 14 to provide flow communication with the internal volume 36 and valve opening 46 for delivery of the treatment fluid 16 to the contact surface 40.

The treatment tip 14 can further include a retaining recess 50 or other structure that facilitates mounting of the treatment tip 14 to the container 12. Other treatment tip constructions are possible for mounting to the container 12 in alternative ways such as, for example, mounting to the outer surface 30 of the container 12 rather than to the inner surface 28 as shown in FIGS. 1-3.

The valve opening 46 can be part of a valve member. The valve member can be defined by structure of the treatment tip 14 or can be a separately constructed device that is mounted to the treatment tip 14 in a separate step. In one example, the valve member is a slit defined between the contact surface 40 and the fluid channel 48. The slit can be configured to open only when a predetermined pressure level is generated within the internal volume 36, thereby permitting flow of the treatment fluid 16 through the slit and to the contact surface 40. The slit can be defined as a one-way valve that limits backflow of fluid or other foreign objects (e.g. skin particles, blood or puss from the acne member) into the internal volume 36.

Many other valve structures besides the slit 46 are possible for use in controlling fluid flow between the internal volume 36 and the container 12 and the contact surface 40 of the treatment tip 14. Typically, whatever valve structure is used will be configured to retain the treatment fluid 16 within the container 12 until such time as the user is ready for the flow of treatment fluid to the contact surface 40.

The treatment tip 14 can comprise a compliant or semi-compliant material, such as, for example, polypropylene, polyolefins (e.g., HDPE, LDPE, and silicone), urethane, fluropolymers (e.g., Teflon), and rubber-like elastomers (e.g., EPDM, BUNA-N, neoprene, latex, rubber, and Viton). The materials for the treatment tip 14, especially in the area of the contact surface 40, can provide for a comfortable interface between the device 10 and the skin surface 18. The material of the treatment tip 14 and the area of the contact surface 40 can also provide for some deforming or otherwise adjustment in size of the treatment tip 14 to adequately enclose the acne member 20 while maintaining sealing engagement with the skin surface 18. Material of the treatment tip 14 typically has sufficient rigidity so as to be maintained mounted to the container 12 without inadvertent removal from the container 12.

The treatment tip 14 may be formed as a separate piece that is mounted to the container 12 in a separate step. In other arrangements, the treatment tip is formed integral with the container 12 as a single piece structure. In one example, a portion of the integral treatment tip and container device, a portion of the treatment tip is removable to permit fluid flow out of the treatment tip. Such integral tip/container devices are sometimes referred to as “single use” devices that are disposable after, for example, a single use.

Referring now to FIGS. 2 and 3, an example method of treating the acne member 20 is described. FIG. 2 illustrates the treatment device 10 aligned with the treatment tip 14 facing the acne member 20 on the skin surface 18. Typically, the treatment device 10 is oriented relative to the skin surface 18 such that the treatment fluid 16 is in engagement with the fluid channel 48 and compression of the container 12 forces the treatment fluid 16 out of the valve opening 46.

After proper alignment is achieved, the device 10 is moved into engagement with the skin surface such that the contact surface 40 of the treatment tip 14 is in sealing engagement with the skin surface 18 at least partially surrounding the acne member 20. At least a portion of the acne member 20 is enclosed within the recess defined by the contact surface 40. Usually, a treatment space 68 is defined between the contact surface 40 and the acne member 20 and sized to retain a volume of treatment fluid.

FIG. 3 illustrates the treatment tip 14 in engagement with the skin surface 18. After a seal is created between the treatment tip 14 and skin surface 18 a compression force either in the direction X, the direction Y, the Z direction, or a combination of compression forces is applied in various directions to create fluid flow out of the container 12 and into the treatment tip 14. The fluid flow may occur due to an increased pressure condition within the internal volume 36. The increased pressure condition within the internal volume 36 may force the treatment fluid 16 out of the valve opening 46 into the treatment space 68 in contact with the acne member 20. In some arrangements, the valve opening 46 is configured to open only after a predetermined increased pressure condition is generated within the internal volume 36. In other arrangements, the valve opening 46 can be opened followed by supplying a pressurized volume of the treatment fluid to the valve opening 46.

The treatment fluid 16 may be applied to the treatment space 68 in a pressurized condition. The treatment fluid can maintain a pressurized condition while in the treatment space 68 and while in contact with the acne member 20 to facilitate penetration of the treatment fluid into the acne member 20. Typically, the acne member is opened in advance of applying the acne treatment fluid. For example, the acne member can be squeezed to burst the acne member thereby expelling some of the puss and fluid retained within the acne member. Bursting the acne member creates a fluid flow path into and out of the acne member. Such a fluid path can increase penetration of the acne treatment fluid into the acne member.

The treatment tip 14 is typically maintained in contact with the skin surface 18 for a predetermined time period to facilitate penetration of the treatment fluid 16, whether or not in a pressurized state, into the acne member 20. The predetermined time period can be, for example, in the range of about 1 second to about 20 seconds, and more preferably in the range of about 2 seconds to about 10 seconds. In some arrangements, the predetermined time period can be longer such as in the range of, for example, 20 seconds to as many as 3 to 5 minutes. The predetermined time period needed for ideal treatment of the acne member can vary depending on a number of variables such as, for example, the pressure level of the treatment fluid within the treatment space 68, adequacy of the sealing engagement between the treatment tip 14 and the skin surface 18 surrounding the acne member, the size and shape of the acne member, the condition of the acne member (i.e., whether the acne member has been opened or not), and the composition of the acne treatment fluid.

In some arrangements, the compression force applied to the container 12 can be released during the predetermined time period. For example, the compression force can be released when the valve opening 46 is part of a one way valve that does not permit back flow of fluid into the internal volume and sealing engagement is maintained between the treatment tip 14 and skin surface. Typically, a force is applied in the direction Y or at other locations on the container 12 to maintain the sealing engagement between the treatment tip 14 and skin surface 18 regardless of whether the compression force is maintained to supply and/or maintain the pressurized volume of treatment fluid to the treatment space 68.

After the predetermined time period, the device 10 can be removed from engagement with the skin surface. Any excess treatment fluid that has not been penetrated into the acne member can be removed from the skin surface 18. The steps of treating the acne member 20 can be repeated by reengaging the treatment tip 14 with the skin surface and supplying a pressurized volume of treatment fluid to the treatment space 68 followed by the lapsing of another predetermined time period while the treatment fluid is maintained in contact with the acne member for improved penetration of the treatment fluid into the acne member.

In some arrangements, it is not possible to maintain adequate sealing engagement between the treatment tip 14 and the skin surface 18 (e.g., at the peripheral edge 42). In such arrangements, the user may need to maintain a continued application of the compression force to supply an ongoing supply of the treatment fluid into the treatment space 68 to maintain contact of the treatment fluid with the acne member for an adequate period of time to ensure some of the treatment fluid enters the acne member while other of the treatment fluid escapes around the peripheral edge 42 and along the skin surface 18.

The treatment fluid can comprise an antiseptic or antibacterial solution such as, for example, super oxygenated water, alcohol, acid, iodine, sodium chloride, saline solution, antibiotic, or a peroxide such as hydrogen peroxide or benzyl peroxide. The treatment fluid typically functions to kill bacteria in the acne member. The treatment fluid can also be used to flush out bacteria and bodily fluids from the acne member.

The treatment fluid can comprise an antihemorrhagic agent such as alum or titanium dioxide that assists in, for example, slowing down bleeding, drying up a wound, or promoting a smooth, healed skin surface. The treatment fluid may comprise medications that reduce excess scabbing of the wound or excess fluid escapement from the wound.

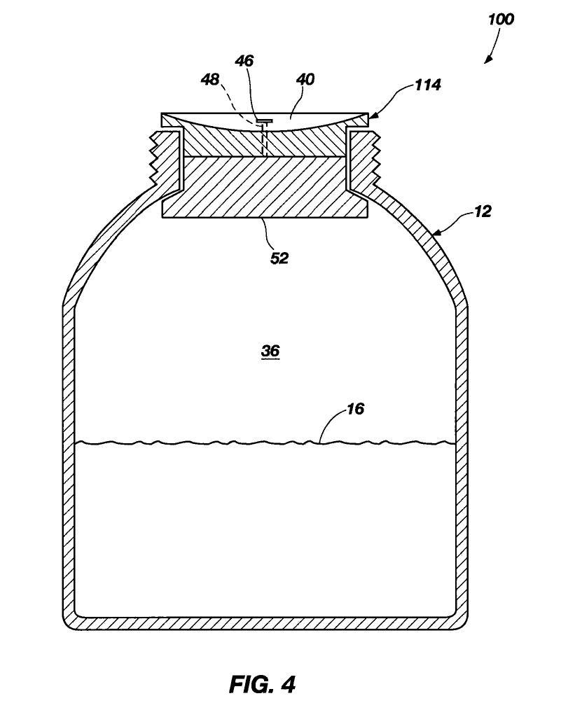

Referring now to FIG. 4, another example treatment device 100 is shown and described. The treatment device 100 includes a container 12 and a treatment tip 114. The container 12 can have many of the same features as described above as referenced to container 12 shown in FIGS. 1-3.

The treatment tip 114 includes a contact surface 40, a peripheral edge 42, a valve opening 46, a fluid channel 48 and a fluid metering and control device 52. The metering and control device 52 is interposed between the valve opening 46 and the treatment fluid 16 retained within the internal volume 36 of the container 12. The metering and control device 52 can be configured to provide many different functions, for example, controlling the amount of treatment fluid 16 delivered to the contact surface 40 at a particular flow rate, a minimum or maximum fluid volume, or a minimum or maximum pressure for the treatment fluid. The fluid metering and control device 52 can comprise any number of mechanical and electrical components such as, for example, valves, sensors, control units and electronics necessary to provide the desired metering and control functions. In one example, the device 52 can comprise bag-on-valve technology to help provide the acne treatment fluid in a pre-pressurized condition.

The metering and control device 52 can be used in conjunction with other acne treatment systems and devices such as the acne treatment system 200 described with referenced to FIG. 5 below. In one example, at least portions of the metering and control device 52 are positioned exterior of the container 12. In some arrangements, the metering and control device 52 is formed integral with the treatment tip 14, such as with at least a portion of the contact surface 40 mounted to or otherwise defined integral with portions of the metering and control device 52. In still other arrangements, at least portions of the metering and control device 52 are mounted to the container 12 or other structure so as to be at least partially removed from and out of engagement with the treatment tip 14.

FIG. 5 illustrates an acne treatment system 200 that includes a treatment tip 14 mounted to a handle 64. The handle 64 includes an activator 66 that controls fluid flow to the treatment tip 14. The system 200 further includes a pressure source 62 that can include a container 12 that retains a volume of treatment fluid 16. The pressure source 62 is coupled in fluid communication with the handle 64 via a supply line 60. In some arrangements, the container 12 and volume of treatment fluid 16 can be mounted directly to the handle 64 as opposed to be positioned remotely, such as in association with the pressure source 62.

The system 200 can supply a volume of the treatment fluid 16 in a pressurized state to the treatment tip 14. The treatment fluid 16 can be supplied via the supply line 60. Alternatively, the supply line 60 can deliver a supply of pressurized gas or other fluid to the handle 64 for pressurizing a volume of treatment fluid at the handle 64 for delivery to the treatment tip 14.

In one example, the pressure source 62 provides pressure at a constant pressure level. In another example, the pressure source 62 provides a pulsating pressure that results in the treatment fluid 16 being delivered to the treatment tip 14 also in a pulse.

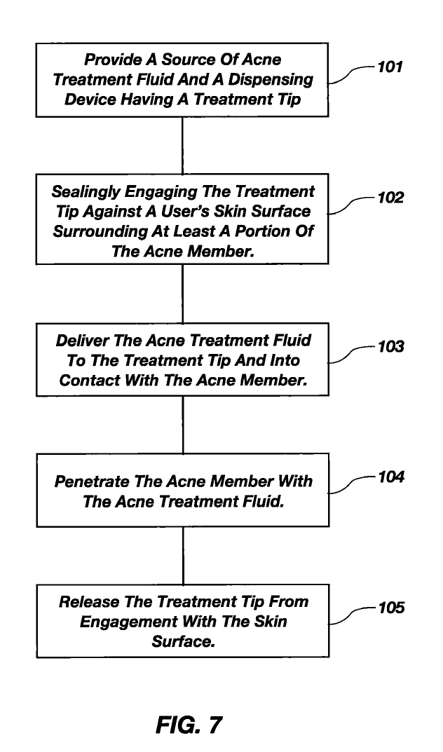

FIG. 7 is flow diagram illustrating example steps in one example method of treating acne in accordance to the principles of the present disclosure. The method includes a step 101 of providing a source of acne treatment fluid and a dispensing device having a treatment tip. A step 102 includes sealingly engaging the treatment tip against a user's skin surface surrounding at least a portion of the acne member. Step 103 includes delivering the acne treatment fluid to the treatment tip and into contact with the acne member. In a step 104, the acne member is penetrated with the acne treatment fluid. A step 105 includes releasing the treatment tip from engagement with the skin surface. The steps of the method shown in FIG. 7 can be repeated as needed to properly treat the acne member by delivering a given amount of treatment fluid into the acne member.

Alternative methods can include more or fewer steps than those shown and discussed above related to FIG. 7. Many different methods are possible in conjunction with each of the embodiments described with reference to the attached figures.

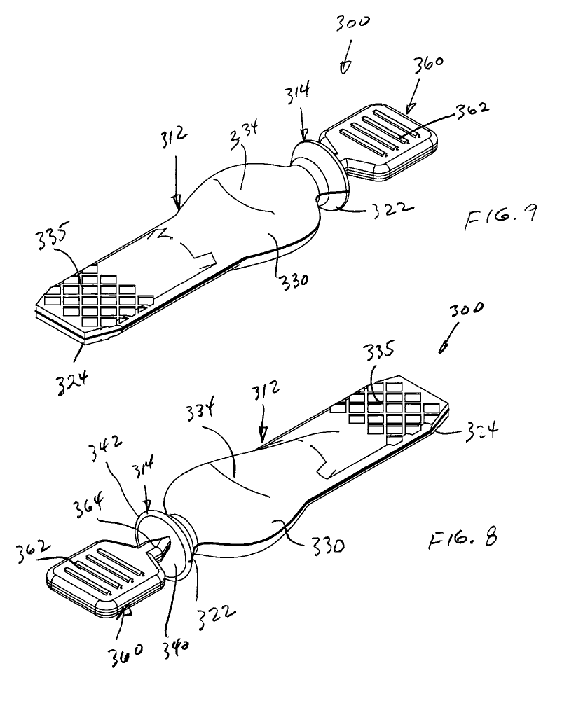

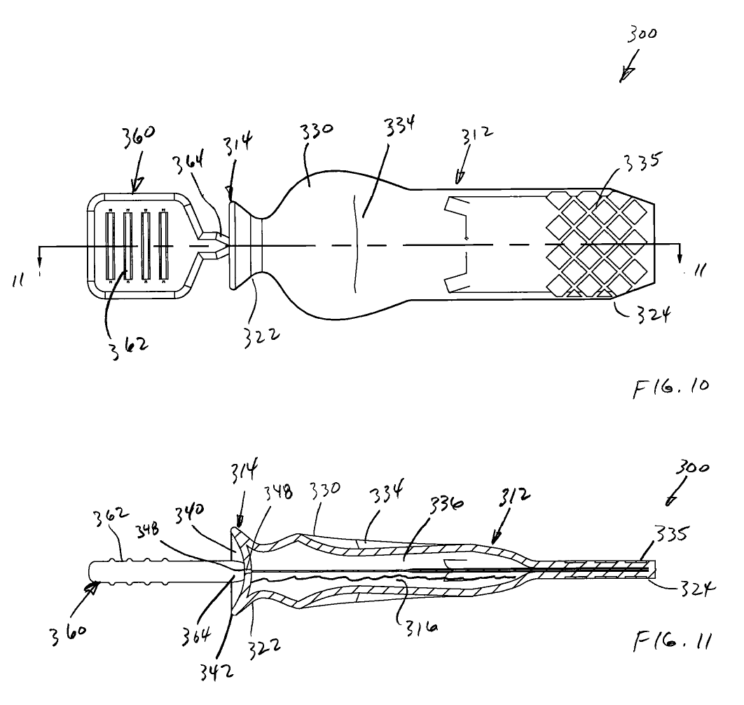

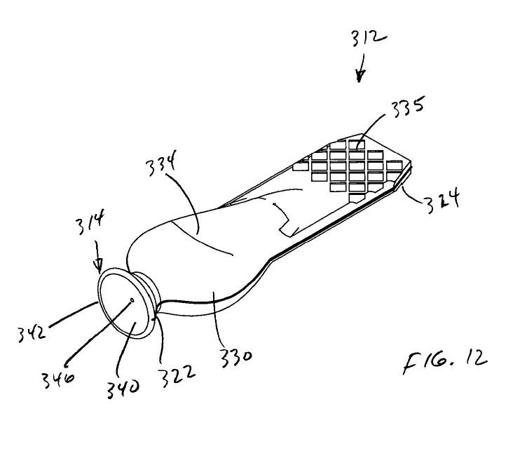

Referring now to FIGS. 8-12, another example treatment device 300 is shown and described. The treatment device 300 includes a container 312 having a treatment tip 314, and a removable cap or tip 360 mounted to the container 312 at the treatment tip 314. Removable cap 360 may be integrally formed with the container 312 (i.e., integrally formed with the treatment tip 314). Removable cap 360 may be configured to remove or disconnect from the container 312 by twisting or pulling removable cap 360 relative to the container 314. Removal of the removable cap 360 exposes an opening into the container 312 that permits fluid held within the container 312 to exit the container 312. Once removed, the removable cap 360 is typically disposed and no longer able to reconnect or be mounted to the container 312 to close the opening. In at least one arrangement, the treatment device 300 is constructed as a disposable device that provides a limited number of uses for treatment of a wound. In one example, the treatment device 300 is configured as a single use device that retains a volume of fluid sufficient to treat a limited number of wounds within a given timeframe or perform a limited number of treatments to a single wound.

The container 312 includes, in addition to the treatment tip 314, a volume of fluid 316 (see FIG. 11), a top or distal end 322, a bottom or proximal end 324, an outer surface 330, and an internal volume 336 wherein the volume of fluid 316 is retained. The outer surface 330 may include a recessed portion 334 and a tab portion 335. The recessed portion 334 may be sized and arranged for improved grasping by the user during application of a compression force using, for example, the thumb and forefinger of the user. The tab portion 335 may provide a grasping surface for easier handling of the container 312. The tab portion 335 may extend away from the internal volume 336 such that grasping the tab portion 335 does not affect a pressure condition within the internal volume 336.

Container 312 may have any size or shape desired. In some arrangements, the container 312 may have a relatively large size that defines an internal volume sufficient to hold a volume of fluid for an extended number of wound treatments. The outer surface may have a generally ergonomic design that promotes easy handling by a user prior to and during use of a treatment device. The container 312 may comprise a plurality of components that are connected together to form a unitary construction. In at least one example, the container comprises two separate halves that are connected together using, for example, an adhesive, heat bonding, or ultrasonic welding to create the unitary structure. In other arrangements, the container 312 is formed using, for example, injection molding, casting or other processes that result in the container being a unitary, single piece device. In at least some examples, the treatment tip 314 is formed unitary with the container 312. In other arrangements, the treatment tip 314 is formed as a separate piece that is connected or otherwise assembled with other components of the container 312 in a separate step.

The treatment tip 314 may include a recess or contact surface 340, a peripheral edge 342 surrounding the recess surface 340, a valve opening 346 positioned in the recess surface 340, and a fluid channel 348 arranged in fluid communication with the valve opening 346. In at least some arrangements, the recess surface 340 is sized to enclose at least a portion of a wound (e.g., a skin acne member). The recess surface 340 may be sized to completely cover or enclose a wound. The peripheral edge 342 is constructed to create a fluid seal or at least a partial fluid seal with a surface such as a skin surface surrounding a wound.

In use, the volume of fluid 316 held in the internal volume 336 of the container 312 passes through the fluid channel 348 and valve opening 346 to a space defined between the recess surface 340 and the wound. Typically, the volume of fluid comes into contact with the wound member or a skin surface through which the wound member is accessible. The volume of fluid 316 can be delivered into the recess defined by the recess surface 340 under an increased pressure condition (i.e., a pressure condition or state that is greater than a rest pressure condition within the internal volume 336). Container 312 may include a compressible or deformable portion (e.g., along the recess 334) on the outer surface 330 to facilitate creating the increased pressure condition within the internal volume 336. Other features can be used in conjunction with the treatment device 300 to create the increased pressure condition or to deliver the volume of fluid 316 from the internal volume 336 to the wound member via the valve opening 346.

The removal cap 360 includes a grasping portion 362 and a sealing portion 364. Typically, the sealing portion 364 is connected to the container 312 adjacent to the valve opening 346 along the recess surface 340. While the removal cap 360 is connected to the container 312, the valve opening 346 maintains a closed state so that the volume of fluid 316 is retained in the internal volume 336. Upon removal of the removal cap 360 by, for example, twisting or pulling the grasping portion 362, the valve opening 346 is exposed so that the volume of fluid 316 can exit the internal volume 336 to treat the wound.

In some arrangements, the treatment device may include a plurality of valve openings along the recess surface 340. A plurality of removable caps may be mounted to the container 312 in association with the plurality of valve openings such that removal of the removable caps exposes individual valve opening. Alternatively, a single removable cap can be used to seal close a plurality of valve openings, wherein removal of the removable cap opens all of the valve openings.

In other arrangements, the treatment device includes a valve opening that automatically opens upon creation of an increased pressure condition in the internal volume. The treatment device may be void of a removal cap or other feature that is physically removed by the user to expose an opening for delivering the volume of fluid. A layer, film, or seal portion may be positioned in or adjacent to the valve opening (e.g., along the recess surface 340) and be configured to automatically open when a predetermined pressure level is attained within the internal volume 336. The predetermined pressure level may be reached by, for example, applying a compression force to the outer surface 330.

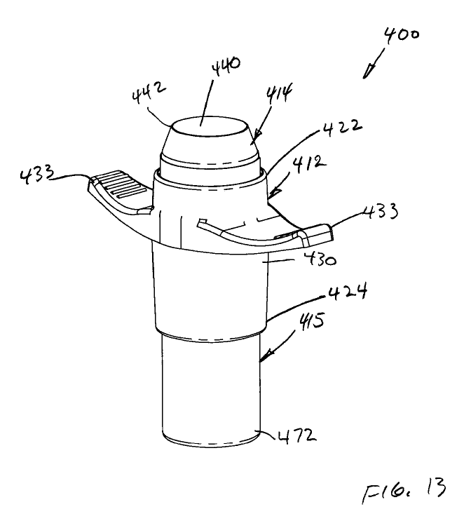

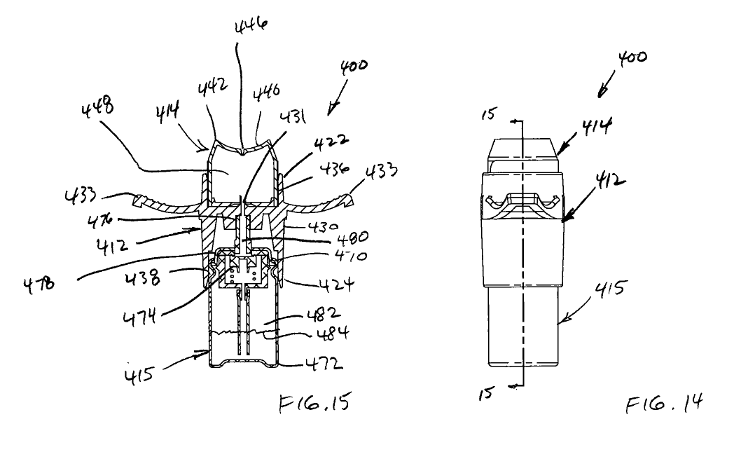

Referring now to FIGS. 13-16, another example treatment device 400 is shown and described. The treatment device 400 includes a housing 412, a treatment tip 414 and a fluid container 415. When assembled, the treatment device 400 is operable by a user to deliver fluid retained within the fluid container 415 through the housing 412 and treatment tip 414 to a wound positioned at least partially within or adjacent to the treatment tip 414.

The housing 412 includes a distal end 422, a proximal end 424, a side surface 430, and a pair of finger grip members 433. The finger grip member 433 may extend from the side surface 430. In some arrangements, the finger grip members 433 are replaced with a single finger grip member 433 or other gripping features. The housing 412 may also include a treatment tip 433 defined in the distal end 422 and sized to receive at least a portion of the treatment tip 414. The housing 412 may also include a fluid container seat 438 positioned at the proximal end 424. The fluid container seat 438 may be sized to receive at least a portion of the fluid container 415. A fluid channel 431 may be defined within the housing 412 for passage of fluid from the fluid container 415 to the treatment tip 414.

Treatment tip 414 includes a recess or a contact surface 440, a peripheral edge 442 defined around at least a portion of the recess surface 440, a fluid opening 446, and a fluid cavity 448. The fluid opening 446 defines a flow path between the fluid cavity 448 and the recess surface 440. As with the other treatments tips disclosed herein, the treatment tip 414 is typically sized to at least partially enclose, cover, or surround at a portion of a wound member, such as a skin acne member. The peripheral edge 442 is configured to create a fluid seal or partial fluid seal with a surface such as a skin surface adjacent to a wound member or an exposed portion of the wound member.

Treatment tip 414 may be removable from the housing 412. Treatment tip 414 may be replaced with a new or different treatment tip when, for example, using the treatment device 400 for a different wound or a different patient. Many different sizes and shapes for the treatment tip 414 are possible, including variations in the size and shape of the recess surface 440 and peripheral edge 442.

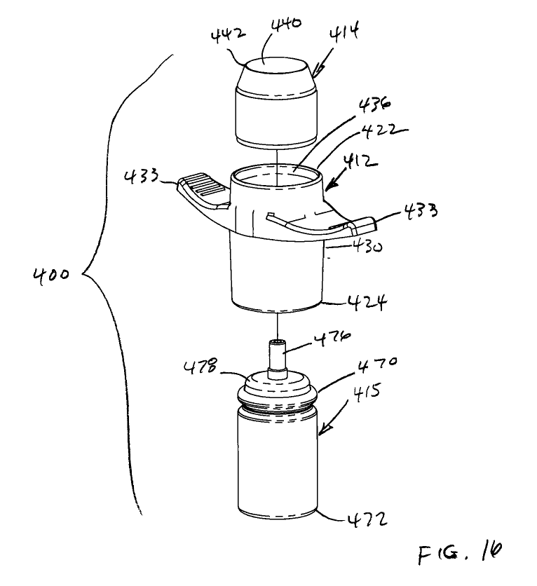

A fluid container 415 may include a distal end 470, a proximal end 472, a valve assembly 474, a dispensing tip 476, a cap 478, a fluid channel 480, and an internal volume 482. The dispensing tip 476 extends distally from the cap 478. The valve assembly 474 (see FIG. 15) is positioned between the dispensing tip 476 and the treatment fluid 484 held within the internal volume 482. Compressing the dispensing tip 476 in the proximal direction actuates the valve assembly 474 to create a fluid flow path for the treatment fluid 486 to exit through the dispensing tip 476 and fluid channel 431 of the housing 412 into the treatment tip 414.

The treatment fluid 484 may be retained in a pressurized state within the internal volume 482. In at least one example, the internal volume 482 retains a propellant in addition to the treatment fluid 484. The propellant mixes with the treatment fluid 484 so that when a fluid flow path is opened through the valve assembly 474 the treatment fluid or combination of treatment fluid and propellant are expelled from the dispensing tip 476 out of the fluid container 415. Any number of propellants and treatment fluids are possible for use in the treatment devices 300, 400 such as those described above with reference to treatment device 10.

The fluid container 415 can be replaced with a different fluid container having a different size, shape, or retaining a different type of treatment fluid or propellant. Various treatment fluids may be used to treat particular types of wound members. In some examples, multiple fluid containers 415 including different treatment fluids may be used with a single treatment device 400 to treat a given wound member. In at least some examples, the treatment device 400 may be used to treat a wound member by creating a fluid seal on or around the wound member with a treatment tip 414, the fluid container 415 is mounted to the housing 412, and the valve assembly 474 is actuated to deliver the treatment fluid 484 to the wound member. While the fluid seal is maintained at the treatment tip 414, the fluid container 415 may be replaced with a different fluid container that includes the same or different treatment fluid and the valve assembly is actuated again to deliver the treatment fluid from the second fluid container for treatment of the wound member.

The valve assembly 474 is typically actuated by the user by contacting at least one finger or thumb member with at least one of the finger grip members 433, and contacting a finger or thumb of the user with the proximal end 472 of the fluid container 415. The user then applies a force along the proximal end 472 in a direction toward the housing 412 (i.e., in a distal direction) to actuate the valve assembly 474.

During use, the user may apply a first force in the distal direction along the proximal end 472 to create the fluid seal on or around the wound member with the treatment tip 414, followed by an increase of the applied force (i.e., a second force) in the distal direction to actuate the valve assembly 474 to deliver the treatment fluid 484 to the wound member at the treatment 414.

Generally, the treatment device 400 may include a removable treatment tip and a removable fluid container that are separately mounted to a housing. In some arrangements, the treatment tip 414 may be integrally formed with or permanently attached to the housing 412 so that the housing 412 and treatment 414 are a single, unitary assembly or piece to which the fluid container is removably mounted. In some arrangements, the fluid container 415 is permanently mounted to the housing 412. In some arrangements, the fluid container 415 can be refilled with additional or different treatment fluid 484. In some arrangements, the fluid container 415 can be refilled while the fluid container 415 is mounted to the housing 412.

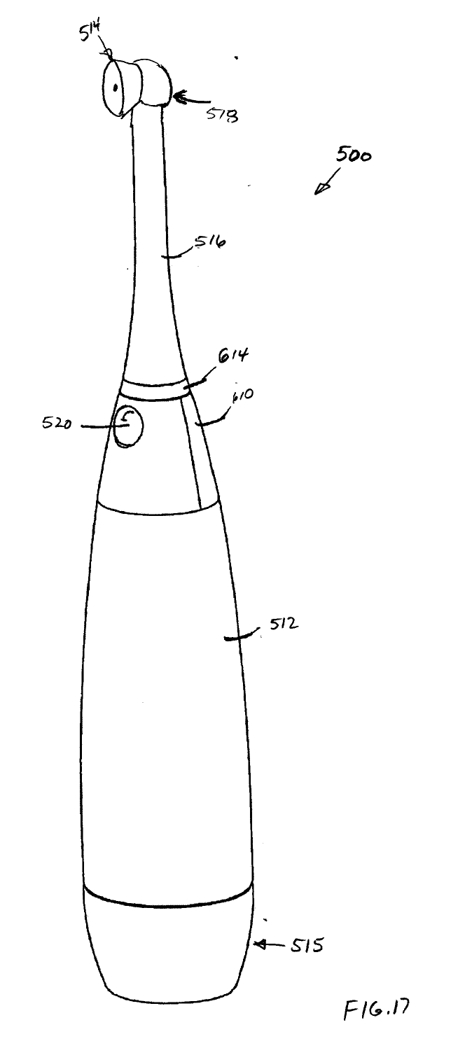

Referring now to FIGS. 17-19, another example treatment device 500 is shown and described. The treatment device 500 includes a treatment tip 514 that may have a similar construction as the treatment tips described above with reference to the figures. The treatment device 500 may include features that provide a source of pressure that pressurizes the treatment fluid delivered by the treatment device 500 at the treatment tip 514. In one example, the treatment device 500 is a hand-held device.

The treatment device 500 may include a self-contained source of treatment fluid. The treatment device 500 may include a power source and other features for a self-contained device (i.e., not connected to any other sources of power or treatment fluid). In other arrangements, the treatment device 500 can be connected to a remote source of fluid via, for example, a fluid hose or conduit that extends from the remote source of fluid and delivers the fluid to the treatment tip. The treatment device 500 may be configured to draw the treatment fluid from the remote source to the treatment tip 514 and deliver the treatment fluid as, for example, a liquid jet stream.

The treatment device 500 may include capabilities to oscillate the pressure or rate of fluid flow expelled at the treatment tip 514. The oscillations in pressure or flow rate may occur at, for example, a frequency in the range for about 1 Hz to about 40 kHz. The oscillating pressure or flow rate of the treatment fluid at the treatment tip 514 may improve the effectiveness of treating the wound as the treatment fluid is agitated or moved when in contact with at least a portion of the wound member or in contact with a skin layer positioned between the treatment tip and the wound.

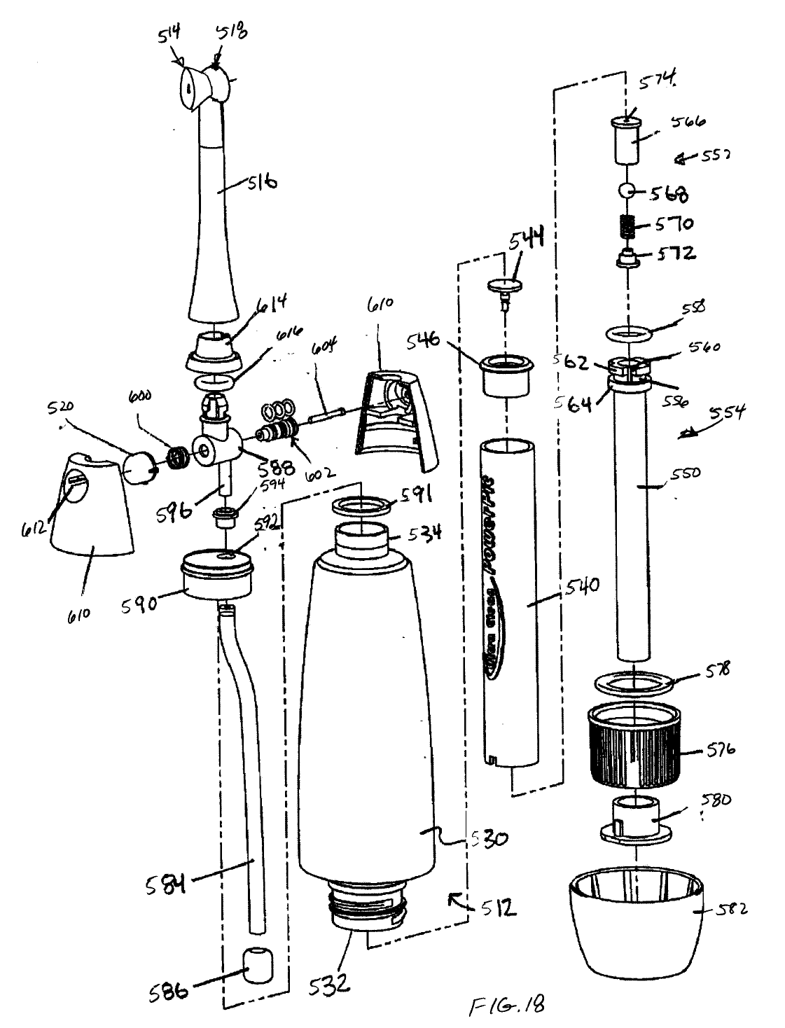

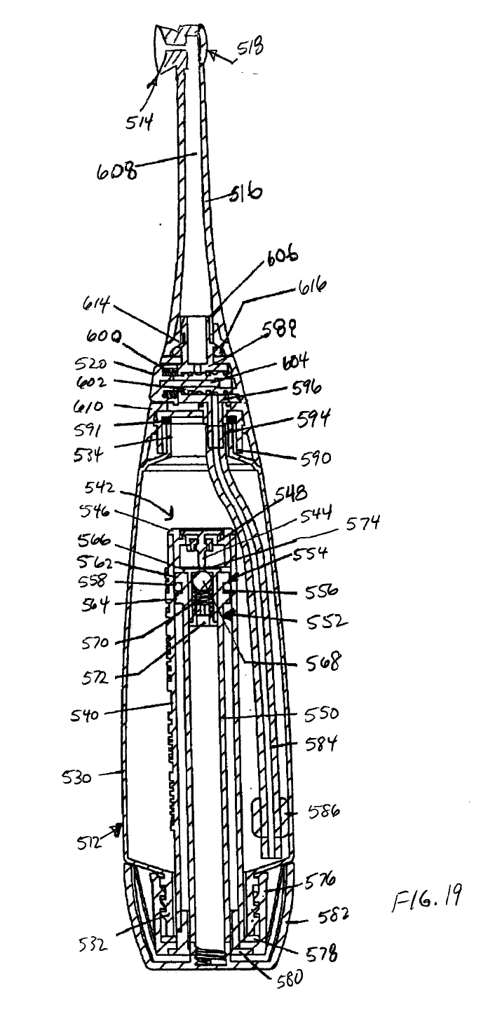

The treatment device 500 includes a body 512 (or handle) that may be gripped by a user. As will be described in detail below, the body 512 is further defined as a refillable reservoir that a user may partially fill with a treatment fluid. The reservoir may retain a treatment fluid that includes a gas, such as air, or a liquid, such as water or an antiseptic solution such as those treatment fluids discussed above. The treatment device 500 also includes a features for pressurizing the fluid in the reservoir. The pressurizing features may include an on-board pump 514. Once the fluid is pressurized, the fluid in the reservoir may be released by depressing a button 520. The button 520 (see FIGS. 18 and 19) opens a valve mechanism which controls the flow of the pressurized fluid out of the reservoir. The fluid, once released, travels through a neck 516 that extends outwardly from the body 512 to a head 518. This neck/head assembly may be interchangeable with other neck/head assemblies to provide the user with different treatment tip configurations 514. Alternatively, the neck/head assembly may also be fixed onto the body 512, to provide a single cleaning implement.

Referring now to FIGS. 18 and 19, the treatment device 500 includes a refillable reservoir 530 that also serves as a main portion of the body 512 or handle that the user grips when using the treatment device 500. The refillable reservoir 530 is attached at one end (e.g., bottom aperture 532) to an on-board pump 515 and attached at the other end (top aperture 34) to the neck 16. Preferably, the on-board pump 515 is attached to the bottom aperture 532 such that the on-board pump 515 may be easily removed and reattached. This permits the user to pour a liquid (or other treatment fluid) into the reservoir 530 via the bottom aperture 532. However, it is also easy to implement alternative means to refill the reservoir such as, but not limited to, having a separate resealable inlet. In this instance, the on-board pump 515 may not have to be removable and could be permanently attached thereto.

The on-board pump 515 includes a pump cylinder 540 that slides into the reservoir 530 through the bottom aperture 532 (see FIG. 19). Secured to one end of the pump cylinder 540 is a one-way valve cap 542. The one-way valve cap 542 allows air to be pumped into the reservoir 530, which permits the user to pressurize the liquid (or fluid) inside the reservoir 30. The one-way valve cap 42 also prevents any liquid (or fluid) inside the reservoir 530 from entering the pump cylinder 540. The one-way valve cap 542 includes a flexible valve seal 544 and a valve cap 546. The valve cap 546 attaches to the end of the pump cylinder 540 and includes openings 548 to permit air to travel through. The flexible valve seal 544 is then attached to the valve cap 546 such that the flexible valve seal 544 covers the openings 548 in the valve cap 546.

When the user is pumping air through the pump cylinder 540, to pressurize the reservoir 530, air in the valve cap 546 travels through the openings 548 pushing and bending the flexible valve seal 544 away from the openings 548, allowing air to enter into the reservoir 530. This also occurs when the pressure inside the reservoir 530 is lower than a maximum pressure pre-defined by a pressure release valve 552 (discussed in greater detail below). As soon as the user stops pumping air through the pump cylinder 540, the pressure in the reservoir 530 pushes against the flexible valve seal 544 and seals the openings 548, preventing liquid in the reservoir 530 from escaping via the one-way valve cap 542.

Inserted into the pump cylinder 540 is a pump piston 550 that includes the pressure release valve 552. The top 554 of the pump piston 550 has a groove 556, which receives a seal 558. The top 554 also has notches 560 (see FIG. 18) running perpendicular to the groove 556. While the pump piston 550 is being pulled away from the pump cylinder 540, the seal 558 moves against the top portion 562 of the groove 556, allowing air to enter the pump cylinder 540, above the top 554 of the pump piston 550, via the notches 560. Subsequently, when pushed into the pump cylinder 540, the seal 558 moves against the bottom portion 564 of the groove 556, preventing air from escaping the pump cylinder 540. As such, when the pump piston 550 is pushed into the pump cylinder 540, air inside the pump cylinder 540, above the top 554 of the pump piston, will be forced into the reservoir 530, again as long as the pressure inside the reservoir 530 is lower than the maximum pressure provided for on the pressure release valve 552.

When the pressure inside the reservoir 530 is greater than the maximum pressure provided for by the pressure release valve 552, the air inside the pump cylinder 540 above the top 554 of the pump piston 550 will vent through the pressure release valve 552. The pressure release valve 552 includes a release housing 566, a release ball 568, a release spring 570, and a release cap 572. The release ball 568 is held by the release spring 570 against a release aperture 574 defined in the release housing 566. When the pressure in the reservoir 530 is greater than the pressure exerted by the release spring 570 on the release ball 568, the air will push against the release ball 568 compressing the release spring 570 permitting the air to vent through the release aperture 574 and into the pump piston 550. Eventually the air will vent out of the device 500 through an aperture (not shown) on the pump handle 582.

The reservoir 530, pump cylinder 540, and pump piston 550 are secured and captured by various end caps that secure them separately to the device 500. The reservoir 530 is removably attached to a reservoir cap 576, which may be removed when filling the reservoir 530 with a liquid. A reservoir gasket 578 is positioned between the reservoir 530 and reservoir cap 576 to prevent liquid (or fluid) inside the reservoir 530 from leaking. The pump cylinder 540 extends through the reservoir gasket 578 and reservoir cap 576 and is secured to a pump cylinder cap 580. Extending through the pump cylinder cap 580 is the pump piston 550, which is attached to a pump handle 582. The user is also prevented from pulling the pump piston 550 entirely out of the pump cylinder 540 because the top 554 of the pump piston 550 is larger than an opening defined in the pump cylinder cap 580.

Since the operation of the on-board pump 514 has already been partially explained, it can be further stated that when in operation a user can remove the on-board pump 514 by separating the reservoir cap 576 from the reservoir 530. This permits the user to partially fill the reservoir 530 with a liquid or other fluid. The reservoir 530 may comprise a clear material to allow the user to view the amount of liquid in the reservoir 530. However, a transparent window may also be used. After partially filling the reservoir 530, the user re-attaches the on-board pump 514 by inserting the pump cylinder 40 into the reservoir 30 and securing the cap 576 to the reservoir 530. The user then can extend the pump piston 550 out of the pump cylinder 540 by holding both the pump handle 582 and the reservoir 530 and pulling the pump handle 582 away from the reservoir 530. Air will then enter the pump cylinder 540 by the top 554 of the pump piston 550. The user then pushes the pump piston 550 back into the pump cylinder 540, forcing air through the one-way valve cap 542 and into the reservoir 530.

Repeatedly pumping air into the reservoir 530 will pressurize the liquid contained therein. When the pressure inside the reservoir 530 is substantially equal to the maximum pressure set by the pressure release valve 552, the air will no longer enter the reservoir 530 but will instead vent out of the pump cylinder 540 through the pressure release valve 552. Once the fluid or liquid inside the reservoir 530 is pressurized the user may release the pressurized liquid through a nozzle located in the head 518 of the treatment device 500.

It may be further stated that the user can alter not only the force of the jetting liquid (or fluid) exiting the device 500 via the treatment tip 514, but also the duration such force is maintained. To alter the force of the jetting liquid (or fluid), the user may learn that for a specific water level inside the reservoir the user must pump air into the reservoir ten times (pressurizing the reservoir to a desired pressure). By reducing the number of times the user pumps air into the reservoir, the desired force of the liquid (or fluid) jetting out of the device 500 will decrease. In addition, the duration the force of the liquid (or fluid) jetting out of the device 500 is directly related to the level of liquid (or fluid) inside the reservoir for a specific pressure. As such, if the user reduces the liquid or (or fluid) level, but maintains the desired pressure inside the reservoir 530, the duration this desired force is maintained will increase. Similarly, if the user increases the liquid (or fluid) level, the duration will decrease. As such it may be preferred to have a fill line on the reservoir 30 that permits the user to fill the reservoir to a predetermined amount. This would provide the user with an optimum force of jetting liquid (or fluid) (when pressurized to the maximum pressure) for an optimum maximum duration. In addition, the secondary fill lines can relate to various head attachments, providing optimum settings for different cleaning tasks.

To transport the liquid (or fluid) from the reservoir 530 to the nozzle, a hose 584 is placed through the top aperture 534 of the reservoir 530 and into the reservoir 530. The hose 584 may contain a weight 586 on the end of the hose placed within the reservoir 530 in order to keep the end of the hose 584 near the bottom of the reservoir 530. The other end of the hose 584 is attached to a releasing/preventing mechanism 588 that when activated allows the pressurized liquid or fluid to travel through the neck 516 and out of the device 500.

The hose 584 is secured through an opening 592 in a top reservoir cap 590 that is secured to the top aperture 534 of the reservoir 530, along with a top reservoir gasket 591 (see FIG. 18). The opening 592 is reinforced with a grommet 594 that places the hose 584 in fluid communication With an inlet 596 defined in the releasing/preventing mechanism 588. The releasing/preventing mechanism 588 also includes a valve piston 602 that may be moved to an open position (allowing pressurized liquid or fluid to travel through) by a button 520. A valve spring 600 exerts a force onto the button 520 and the valve piston 602 that normally keeps the releasing/preventing mechanism 588 in a closed position (not allowing pressurized liquid or fluid to travel through). A valve pin 604 holds the button 520 in position with the valve piston 602 and valve spring 600. The releasing/preventing mechanism 88 and other components described for opening and closing the mechanism 588 is housed within a two piece housing 610. The front portion of the housing 610 includes an opening 612 to permit the button 520 to be pressed by a user. As mentioned, the releasing/preventing mechanism 588 is in a closed position unless the button 520 is pressed and held down by a user.

When opened the pressurized liquid or fluid travels through the releasing/preventing mechanism 588 and out an exit 606 defined thereon. The exit 606 of the releasing/preventing mechanism 588 is in fluid communication with a channel 608 running through the neck 516. The neck 516 is secured to a neck base 614 that is removably attached to the exit 606. A neck o-ring 616 may be positioned between the neck base 614 and the two piece housing 610. The channel 608 travels through the neck 516 to the treatment tip 514 on the head 518. The treatment tip 514 may include a valve opening that is in communication with the channel 608.

As explained above, the treatment device 500 includes the ability to jet out a pressurized fluid, not only inclusive of a liquid but also a gas. For example, the user may simple pump air into and pressurize the air inside the reservoir. Once the reservoir contains a sufficient amount of pressurized air, the user may release it by pressing the button. While not as efficient as expelling pressurized liquid, in some instances the liquid, especially an antiseptic liquid, may be too sensitive for the user. Moreover, if pressurized gas such as air was the only intentional use of the device, the pump does not have to be removable, as the user can continuously refill the reservoir with air without removing the pump. The device 500 may oscillate the pressure of the fluid being delivered to agitate the fluid at the treatment tip 514 (i.e., the fluid positioned between a recessed outer surface of the treatment tip and a wound member). Agitating the treatment fluid in contact with the wound member may help penetrate the treatment fluid into the wound member.

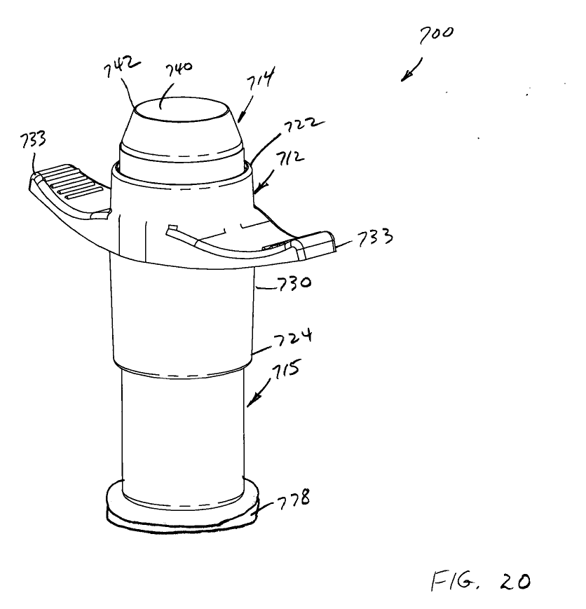

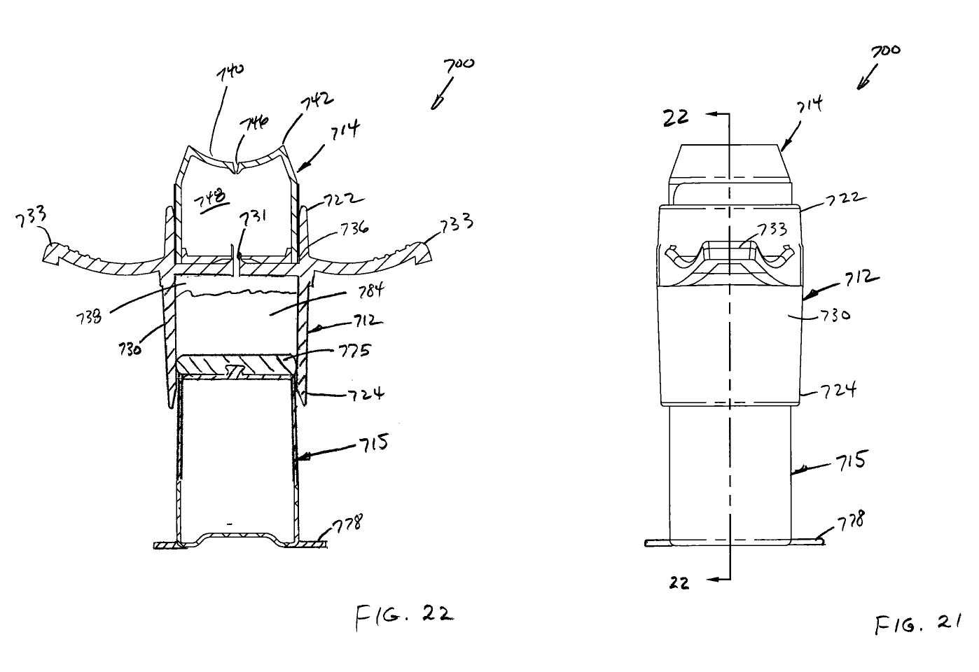

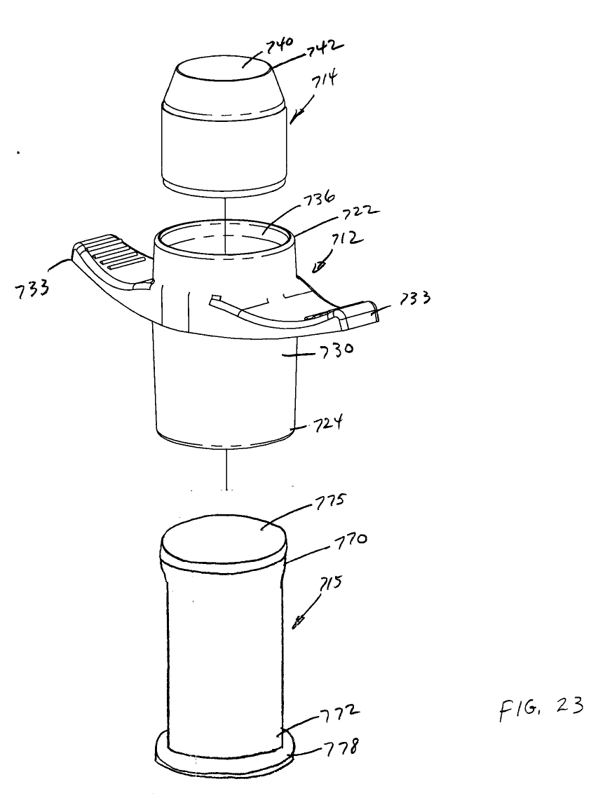

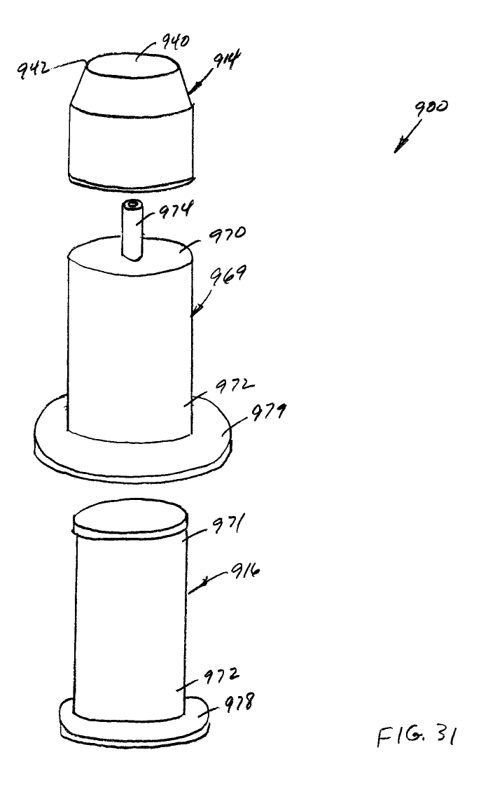

Referring now to FIGS. 20-31, several additional treatment device embodiments 700, 800, 900 are shown and described. The treatment devices 700, 800, 900 each include a plunger feature that assists in delivering the treatment fluid to the treatment tip for treating a wound.

Treatment device 700 shown with reference to FIGS. 1-23 includes a housing 712, a treatment tip 714, and a plunger 715. The housing 712 includes a distal end 722, a proximal end 724, a side surface 730, and a pair of finger grip members 733 extending from the side surface 730. The housing 712 also includes a treatment tip seat 736 within which the treatment tip 714 is positioned, and a fluid cavity 738 that retains a volume of fluid and a portion of the plunger 715. A fluid channel 731 is defined between the treatment tip seat 736 and the fluid cavity 738 to provide fluid flow from the fluid cavity 738 to the treatment tip 714.

The treatment tip 714 includes a recess or contact surface 740, a peripheral edge 742, a fluid opening 746, and a cavity 748. Fluid delivered to the fluid cavity 748 passes out of the treatment device 700 to the recess surface 740 via the fluid opening 746. The peripheral edge 742 helps create and maintain a seal or at least a partial seal between the treatment tip 714 and a contact surface (e.g., a portion of a wound or a surface surrounding the wound).

The plunger 715 includes a distal end 770, a proximal end 772 and a sealing member 775 positioned at the distal end 770. The sealing member 775 may be sized and configured to create a fluid tight seal with an inner surface of the fluid cavity 738 of the housing 712. A flange 778 may be positioned at the proximal end 772 of the plunger 715 for improved ease in contacting and applying a force to the plunger 715 by the user.

In operation, the user contacts the peripheral edge 742 of the treatment tip 714 against a surface and applies a force that creates a seal or at least a partial seal with the contact surface. The user then positions at least one finger or thumb on one of the finger grip members 733 and applies a force to the plunger 715 at the proximal end 772 in the distal direction to deliver a treatment fluid 784 into the treatment tip 714. The treatment fluid 784 travels through the fluid opening 746 to an area (i.e., recess defined by the recess surface 740) adjacent to the recess surface 740. The treatment fluid 784 is delivered to the wound for treatment of the wound that is positioned adjacent to the recess surface 740. In at least some arrangements, the treatment fluid 784 is maintained in contact with the wound for a minimum amount of time while a seal is maintained at the peripheral edge 742.

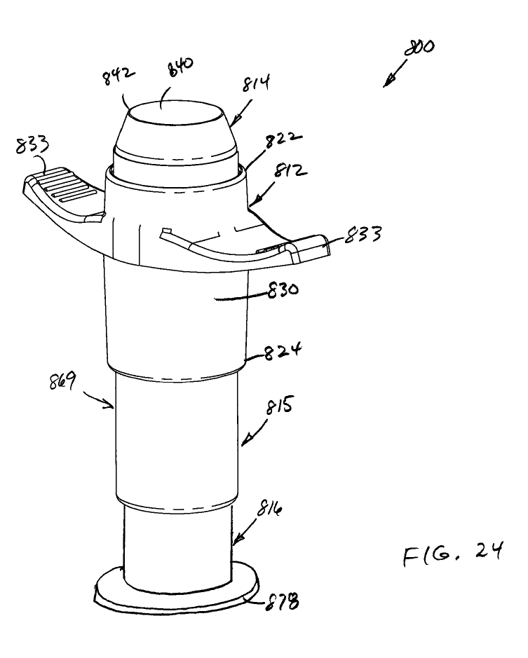

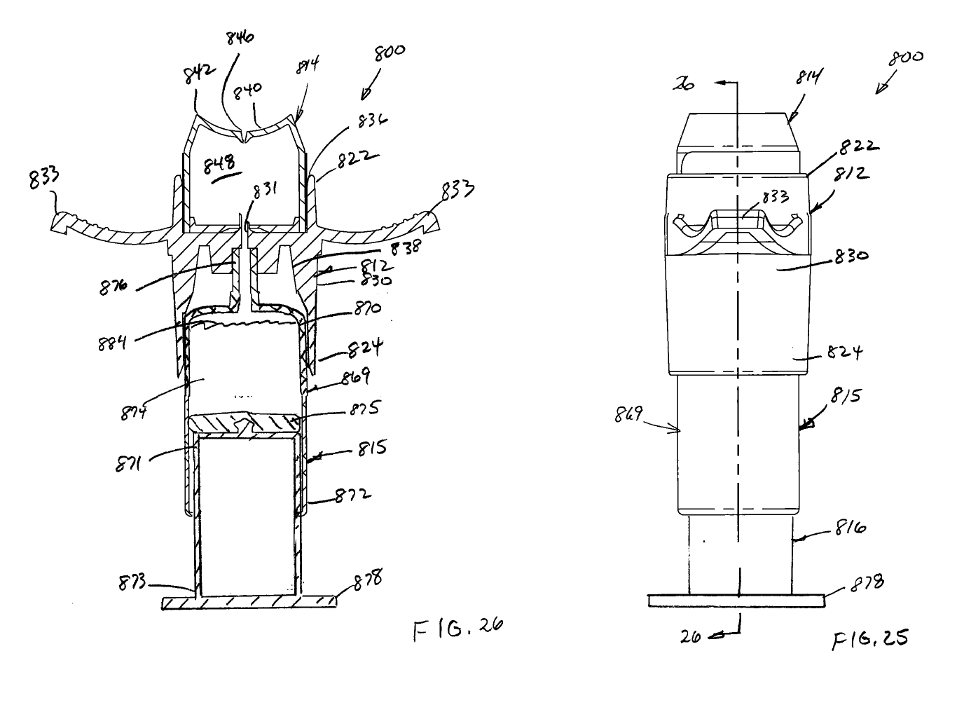

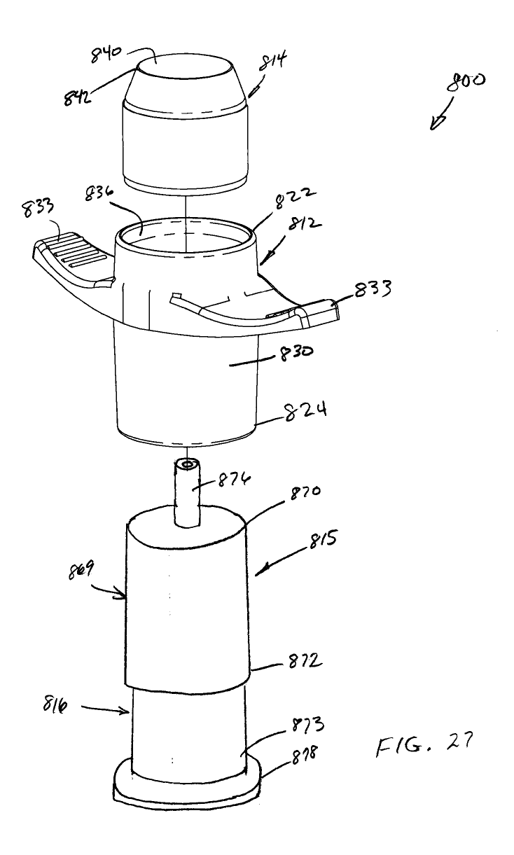

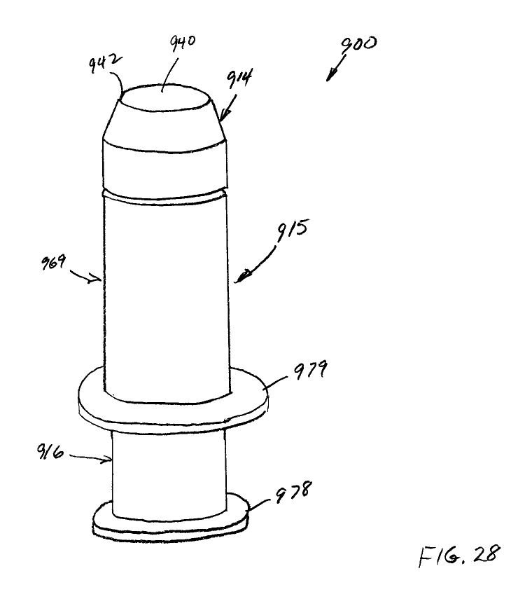

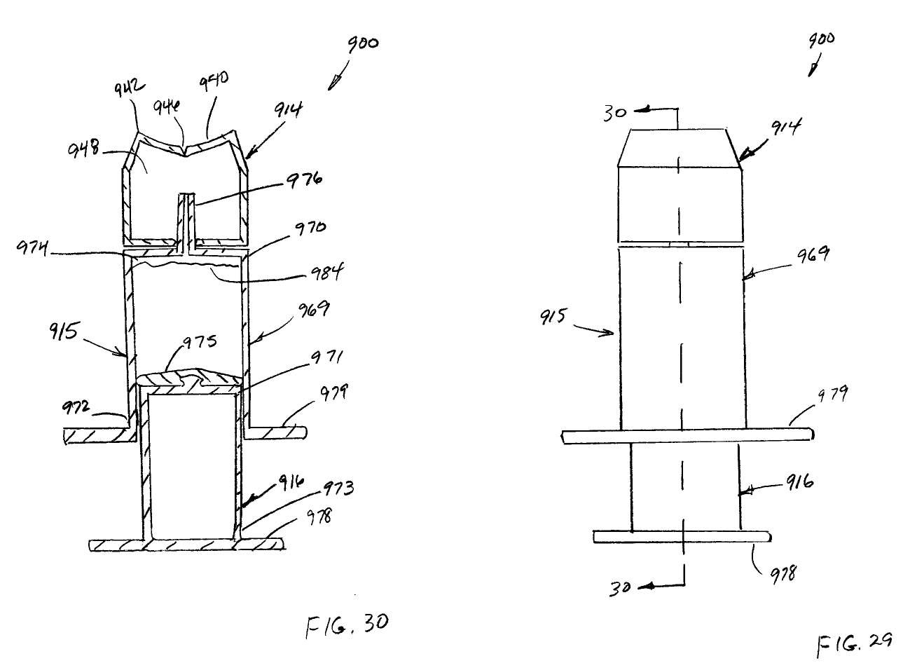

Referring now to FIGS. 24-27, another example and treatment device 800 is shown and described. The treatment device 800 includes a housing 812, a treatment tip 814, and a syringe 815. The syringe 815 mounts to the housing 712. The syringe 815 is operable to deliver a treatment fluid 884 to the treatment tip 814.

The housing 812 includes distal and proximal ends 822, 824, a side surface 830, and at least one finger grip member 833 extending from the side surface 830. The housing 812 may also include a treatment tip seat 836, a syringe seat 838, and a fluid channel 831. The fluid channel 831 may be sized to receive a portion of the syringe 815 for use in delivering the treatment fluid 884 to the treatment tip 814. The treatment tip seat 836 may be sized to receive at least a portion of the treatment tip 814.

The treatment tip 814 includes a recess or contact surface 840, a peripheral edge 842, a fluid opening 846, and a fluid cavity 848. The housing 812 and treatment tip 814 may have features and functionality similar to those housing and treatment tips described above with reference to, for example, treatment device 400.

The syringe 815 may include a body portion 869 and a plunger portion 816. The body portion 869 includes a distal end 870, a proximal end 872, and a cavity 874 that is sized to receive at least a portion of the plunger 816. The body 869 may also include a dispensing tip 876 positioned at the distal end 870.

The plunger 816 may include distal and proximal ends 871, 873 and a sealing member 875 positioned at the distal end 871. A flange 878 may be positioned at the proximal end 873 to assist in moving the plunger 816 axially relative to the housing 812. The cavity 874 of the body 869 may be sized to receive the at least a portion of the plunger 816 (e.g., the sealing member 875). The treatment fluid 884 may be positioned within the cavity 874. Advancing the plunger 816 in the distal direction may deliver the treatment fluid 884 through the dispensing tip 876 and into the treatment tip 814. The delivered treatment fluid 884 may be used to treat a wound that is positioned adjacent to the recess surface 840.

The syringe seat 838 may be sized and constructed to interface with any desired syringe construction. Syringes of various size and fluid capacity may be used with the housing 812. The fluid channel 831 may be sized to interface with a common size for the dispensing tip 876 of known syringes.

Other types of syringe construction may be useful with the treatment device 800. For example, oral syringes having plungers with sealing members integrally formed with the plunger as a single piece may be used rather than a plunger with the sealing member provided as a separate piece having a different material properties (i.e., a rubber sealing member mounted to a plastic plunger body).

Another example treatment device 900 is shown and described with reference to FIGS. 28-31. The treatment device 900 includes a treatment tip 914 configured for mounting directly to a syringe 915 without an intervening structure such as the housing 812 described above. The treatment tip 914 may have the same or similar construction to those treatment tips 414, 714, 814 described above. The treatment tip 914 may be configured to directly receive a dispensing tip 976 of the syringe 915. A fluid tight seal may be provided at an interface between the treatment tip 914 and the syringe 915.

The treatment tip 914 may include a recess or contact surface 940, a peripheral edge 942, a fluid opening 946, and a fluid cavity 948. The size and construction of the treatment tip 914, such as, for example, diameter of the peripheral edge 942 and the depth of the recess surface 940, may be modified for different applications, sizes of wounds being treated, and constructions of the syringe to which the treatment tip 914 is mounted. In some arrangements, the treatment tip 914 may include additional sealing features that include a fluid tight seal with the syringe to which the treatment tip 914 is mounted. In some arrangements, the treatment tip 914 includes a connection feature, such as, for example, a threaded bore that interface with threads on an outer surface of the dispensing tip of the syringe.

This syringe 915 includes a body 969 having distal and proximal ends 970, 972, and a cavity 974. A dispensing tip 976 is mounted to the distal end 970. The syringe 915 also includes a plunger 916 having distal and proximal ends 971, 973 and a sealing member 975 mounted to the distal end 971. The plunger 916 is inserted into the body 969 with the sealing member 975 arranged and configured to create a fluid tight seal with an inner surface of the cavity 974. Advancing the plunger 916 relative to the body 969 in the distal direction delivers a treatment fluid 984 out of the dispensing tip 976 and into the treatment tip 914. The treatment fluid 984 is then delivered through the treatment tip 914 to a wound for treatment of the wound.

Each of the body 969 and plunger 916 may include flange portions 979, 978, respectively, that assist the user in providing relative axial movement between the plunger 916 and the body 969 to deliver the treatment fluid 984 to the treatment tip 914.

The example treatment devices 700, 800, 900 described above with reference to FIGS. 24-31 can include any of the same or similar features as described with reference to the other embodiments shown and described with reference to FIGS. 1-23. The materials used for the treatment device is shown and described with reference to FIGS. 1-23 may be used with any of the examples treatment devices disclosed herein. Furthermore, the treatment fluids and methods of treating a wound as described herein may be generally applicable to all of the treatment device examples without limitation.

The example treatment devices disclosed herein may be modified for use with other wound treatment devices and systems. For example, the treatment tip constructions disclosed herein may be integrated into other treatment fluid delivery devices for internal or external use in treating a wound. In one example, a device similar to the treatment tip described with reference to FIGS. 20-31 may be mounted directly to a dispensing tip of an aerosol canister that contains a treatment fluid. The example treatment tips may also be mounted directly to a lumen or other fluid delivery channel or lumen that is connected to a source of treatment fluid. Any alternative arrangements are contemplated within the scope of the present disclosure.

Various inventive concepts have been described herein with reference to certain specific embodiments and examples. However, they will be recognized by those skilled in the art that many variations are possible without departing from the scope and spirit of the inventions disclosed herein, in that those inventions set forth in the claims below are intended to cover all variations and modifications of the inventions disclosed without departing from the spirit of the inventions. The terms “including” and “having” as used in the specification and claims shall have the same meaning as the term “comprising.”

What is claimed is:

1. A method of treating a wound with a treatment fluid, wherein at least a portion of the wound is positioned below an outer skin surface, the method comprising:
creating a seal on the outer skin surface;
delivering the treatment fluid into the wound;
maintaining the seal to retain the treatment fluid within the wound.

2. The method of claim 1, further comprising a treatment device having a treatment tip configured to provide the seal, wherein delivering the treatment fluid to the wound includes delivering the treatment fluid through the treatment tip.

3. The method of claim 1, further comprising a treatment device that includes a container, wherein the container retains a volume of the treatment fluid, and delivering the treatment fluid includes compressing a portion of the container to increase a pressure condition of the treatment fluid.

4. The method of claim 1, further comprising a treatment device that includes a fluid conduit and a treatment tip, wherein creating the seal includes contacting the treatment tip against the outer skin surface, and delivering the treatment fluid into the wound includes delivering the treatment fluid through the fluid conduit to the treatment tip.

5. The method of claim 1, further comprising mixing the treatment fluid with a propellant prior to delivering the treatment fluid to the wound.

6. The method of claim 1, wherein delivering the treatment fluid includes controlling flow of treatment fluid with a one-way valve member.

7. A wound treatment device, comprising:
a housing configured to retain wound treatment fluid;
a treatment tip including a recess, a peripheral edge surrounding the recess, and a fluid channel in fluid communication with the recess and the volume of wound treatment fluid;
wherein the recess is sized to retain a volume of the wound treatment fluid when the peripheral edge is sealingly engaged with a skin surface surrounding at least a portion of a wound.

8. The wound treatment device of claim 7, further comprising a one-way valve structure arranged between the fluid channel and the recess.

9. The wound treatment device of claim 8, wherein the one-way valve structure includes a slit formed in a portion of the recess.

10. The wound treatment device of claim 7, wherein the housing includes a compressible portion which when compressed increases a pressure state of the wound treatment fluid.

11. The wound treatment device of claim 7, further comprising a source of pre-pressurized propellant configured to expel the wound treatment fluid from the housing into the recess.

12. The wound treatment device of claim 7, wherein the housing and the treatment tip are constructed as a single, unitary piece.

13. The wound treatment device of claim 12, wherein the pressurizing member at least one of a pump, an aerosol source, a bag-on-valve assembly, a compressor, a turbine, and a solenoid.

14. A method of treating a skin acne member, the method comprising:
providing a source of acne treatment fluid and a dispensing device having a treatment tip;
sealingly engaging the treatment tip against a user's skin surface surrounding at least a portion of the acne member;
thereafter delivering the acne treatment fluid to the treatment tip and into contact with the acne member;
penetrating the acne member with the acne treatment fluid.

15. The method of claim 14, wherein delivering the acne treatment fluid includes manually squeezing a portion of the dispensing device.

16. The method of claim 14, wherein delivering the acne treatment fluid includes providing a pressurized source of propellant that mixes with the acne treatment fluid.

17. The method of claim 14, wherein the dispensing device includes a housing and a one-way valve, the housing being sized to retain the acne treatment fluid before delivering the acne treatment fluid, and the one-way valve being positioned between the housing and the treatment tip, and delivering the acne treatment fluid includes passing at least some of the acne treatment fluid through the one-way valve.

18. The method of claim 14, wherein the treatment tip includes a recessed portion, and the step of sealingly engaging the treatment tip against the user's skin surface includes positioning at least a portion of the acne member within the recessed portion.

19. The method of claim 14, wherein the treatment tip includes a circular outer periphery and a concave recessed portion, and the step of sealingly engaging the treatment tip against the user's skin surface includes positioning the acne member within the recessed portion.

20. The method of claim 14, further comprising after the step of delivering the acne treatment fluid, maintaining the sealing engagement of the treatment tip against the user's skin surface for at least 2 seconds.

21. The method of claim 14, further comprising prior to the step of sealingly engaging the treatment tip against the user's skin surface, creating an opening into the acne member through the user's skin surface.

22. The method of claim 14, wherein delivering the acne treatment fluid includes oscillating at least one of a flow rate and a pressure of the acne treatment fluid.
